(19) United States
(12) Patent Application Publication
(10) Pub. No.: US 2016/0256182 A1
(43) Pub. Date: Sep. 8, 2016

(54) MEDICAL INSTRUMENT

(71) Applicants: Johann Klaffenbock, Strobl (AT); Lukas Klaffenbock, Graz (AT); Julian Mair, Munchen (DE)

(72) Inventors: Johann Klaffenbock, Strobl (AT); Lukas Klaffenbock, Graz (AT); Julian Mair, Munchen (DE)

(21) Appl. No.: 14/442,572

(22) PCT Filed: Nov. 12, 2013
(86) PCT No.: PCT/EP2013/073592

§ 371 Date: May 13, 2015

(30) Foreign Application Priority Data
Nov. 13, 2012 (AT) A 50509/2012

(51) Int. Cl. A61B 17/28; F15B 15/14

(52) U.S. Cl. CPC A61B 17/282; F15B 15/14; A61B 2017/00539

(57) ABSTRACT

The invention relates to a medical instrument with an elongated element and an end effector (200) arrangeable at one end of the elongated element. The end effector (200) is operable by means of a hydraulic actuating mechanism (100), and the hydraulic actuating mechanism (100) has a cylinder body (110) with at least one, preferably two, three, four or five, overflow channels (112a, 112b, 112c) for a hydraulic fluid, the overflow channels being connected via at least one bore to the interior of the cylinder body (110). A piston (140) movable along the longitudinal axis (A) of the cylinder body (110) is provided in the cylinder body (110). The piston (140) subdivides the interior of the cylinder body (110) into a first cylinder chamber and a second cylinder chamber and is connected via a piston rod (141) to the end effector (200). At least one connection (123) for introducing hydraulic fluid into the first cylinder chamber and at least one further connectinion (124a, 124b, 124c, 124d, 124e), connected to at least one first overflow channel (112a, 112b, 112c) of the cylinder body (110) are provided, and at least one passage (130) for a surgical or optical instrument, the longitudinal axis of which substantially coincides with the longitudinal axis (A) of the cylindrical body (110), is arranged in the interior (113) of the cylinder body (110).

The invention relates to a medical instrument with an elongated element and an end effector arrangeable at one end of the elongated element, wherein the end effector is operable by means of a hydraulic actuating mechanism, the hydraulic actuating mechanism has a cylinder body with at least one, preferably two, three, four or five overflow channels for a hydraulic fluid, which are connected via at least one bore to the interior of the cylinder body, a piston arranged in the cylinder body and movable along the longitudinal axis of the cylinder body is provided, wherein the piston subdivides the interior of the cylinder body into a first cylinder chamber and a second cylinder chamber and is connected via a piston rod to the end effector, and at least one connection for introducing hydraulic fluid into the first cylinder chamber and at least one further connection which is connected to at least one overflow channel of the cylinder body are provided.

U.S. Pat. No. 4,485,817 A describes a hydraulic actuating mechanism for an end effector in which a piston is moved against a second in order to build up the pressure in the hydraulic fluid required for actuation of the end effector. A disadvantage with this hydraulic actuating mechanism is in particular its complicated structure and the fact that its actuating mechanism allows the triggering of only two defined operating states of the end effector. In particular in gastric plication however, frequently more than two operating states of the end effector are required so that the actuating mechanism is unsuitable for such end effectors. A similar device is also disclosed in U.S. Pat. No. 5,361,583 A.

AT 507.653 B1 discloses the medical instrument described above where the end effector described here has two gripping elements with which, for example, the tissue is fixed in the course of a gastric plication.

In order to ensure that the two gripping elements of the end effector intervene at the correct point in the tissue, the position of the end effector on the tissue is usually fixed with the aid of tissue intervention elements which, for example, are configured to be corkscrew-shaped before the two gripping elements of the end effector are moved towards one another to perform the plication.

It is now the object of the present invention to further develop the hydraulic actuating mechanism described in AT 507.653 B1 such that this allows the use of additional surgical and/or endoscopic instruments.

This object is solved according to the invention whereby at least one passage for a surgical and/or optical and/or therapeutic instrument is arranged in the interior of the cylinder body, the longitudinal axis whereof preferably substantially coincides with the longitudinal axis of the cylinder body.

In a preferred embodiment of the invention, the passage is arranged in the piston rod. Thus, an additional surgical instrument or however also an optical unit such as, for example, an endoscope can be guided through the medical instrument according to the invention in a space-saving manner. It is particularly preferably provided that the surgical instrument is a tissue intervention element, preferably having a corkscrew-like tip.

In a particularly preferred embodiment of the invention, the at least one overflow channel has a first section which runs as a recess on the outer surface of the cylinder body substantially normally to the longitudinal axis of the cylinder body and is connected via at least one bore to the interior of the cylinder body. Furthermore said at least one overflow channel has a second section which runs as a recess on the outer surface of the cylinder body substantially parallel to the longitudinal axis of the cylinder body and said at least one overflow channel has a third section in the base of the cylinder body which is connected to a connection for the hydraulic fluid, As a result of this embodiment of the at least one overflow channel, a particularly space-saving arrangement is obtained in which the connection for the supply line of the hydraulic fluid is arranged in the base of the cylinder body where the cylinder base in one embodiment of the invention is designed as a sealing disk which can be arranged on the cylinder body. In another embodiment, the cylinder base is fabricated in one piece with the cylinder body. The special design of the at least one overflow channel in particular allows a more precise control of the hydraulic actuating mechanism and therefore an improved actuation of the end effector.

In addition, it is particularly preferably provided that the at least one bore of the first section of the at least one overflow channel is arranged between an upper and a lower piston stop so that the piston inside the cylinder body can be brought into at least one defined middle position which is arranged between the upper and the lower piston stop.

The use of the medical instrument according to the invention is not restricted merely to gastric plication. Thus, most diverse end effectors can be attached, particularly preferably the end effector in this case has two claw-like gripping elements which can be moved towards one another from an open position into at least one closed position, preferably into two closed positions.

The invention is explained in detail hereinafter with reference to non-restrictive exemplary embodiments with relevant figures. In the figures:

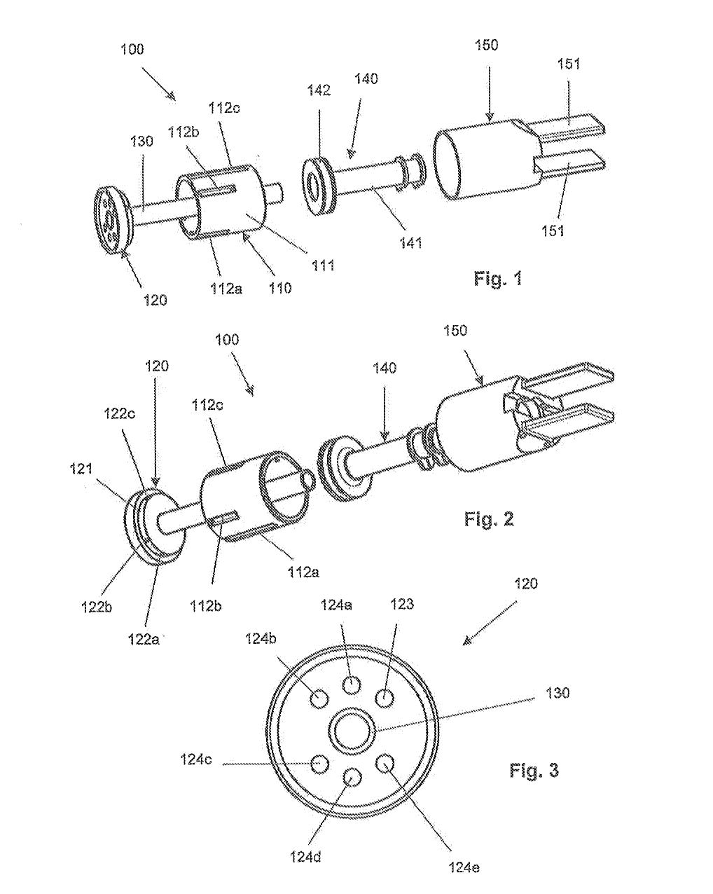

FIG. 1 and FIG. 2 each show an exploded view of the hydraulic actuating mechanism in a first embodiment;

FIG. 3 shows a plan view of the cylinder base from FIG. 1;

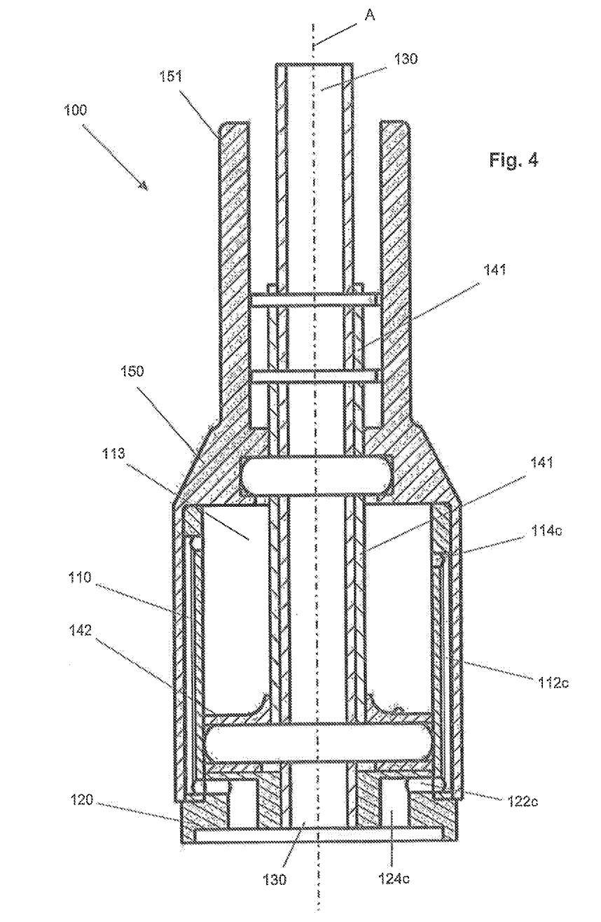

FIG. 4 shows a sectional view of the hydraulic actuating mechanism in the assembled state from FIG. 1;

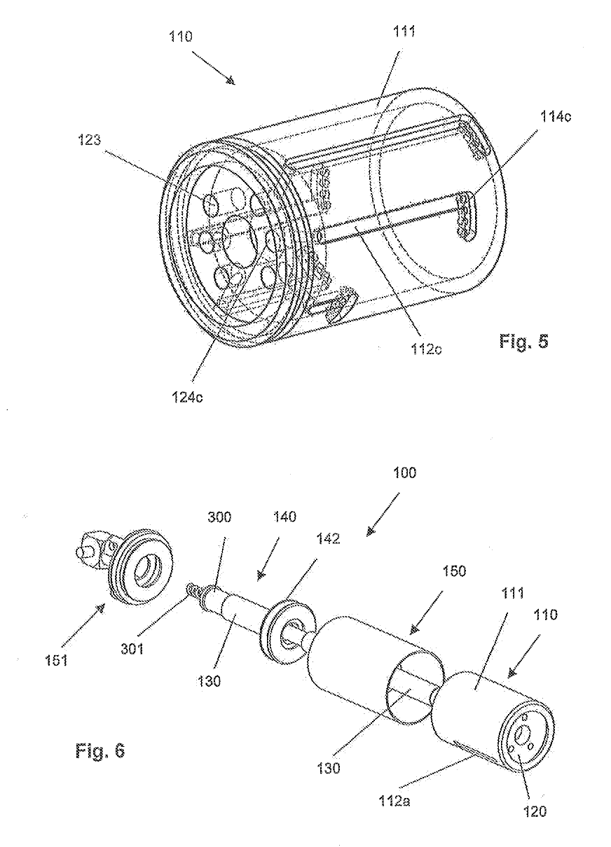

FIG. 5 shows an oblique view of the cylinder body from FIG. 1;

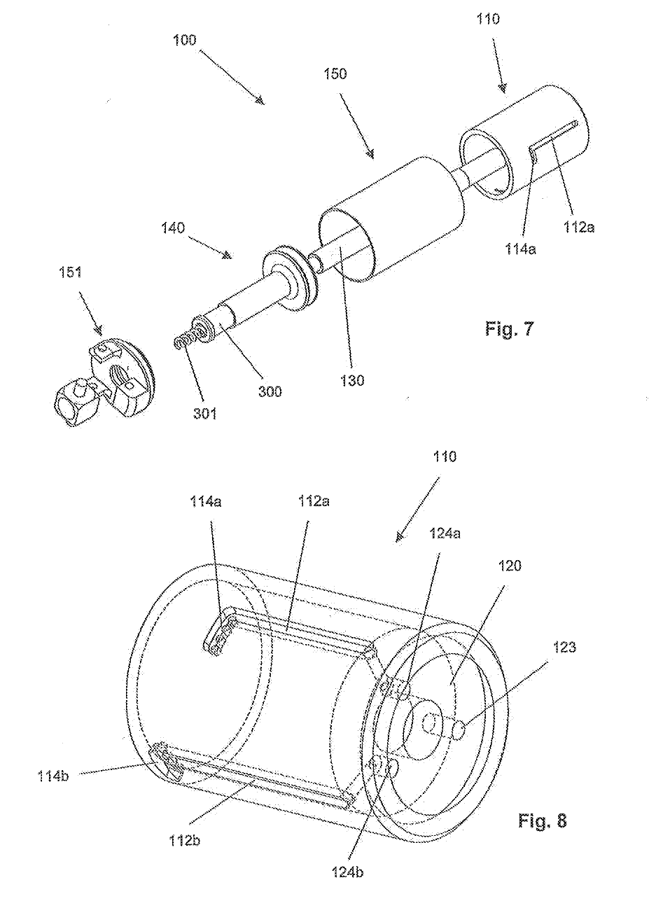

FIG. 6 and FIG. 7 each show an exploded view of the hydraulic actuating mechanism in a second embodiment;

FIG. 8 shows an oblique view of the cylinder body from FIG. 6;

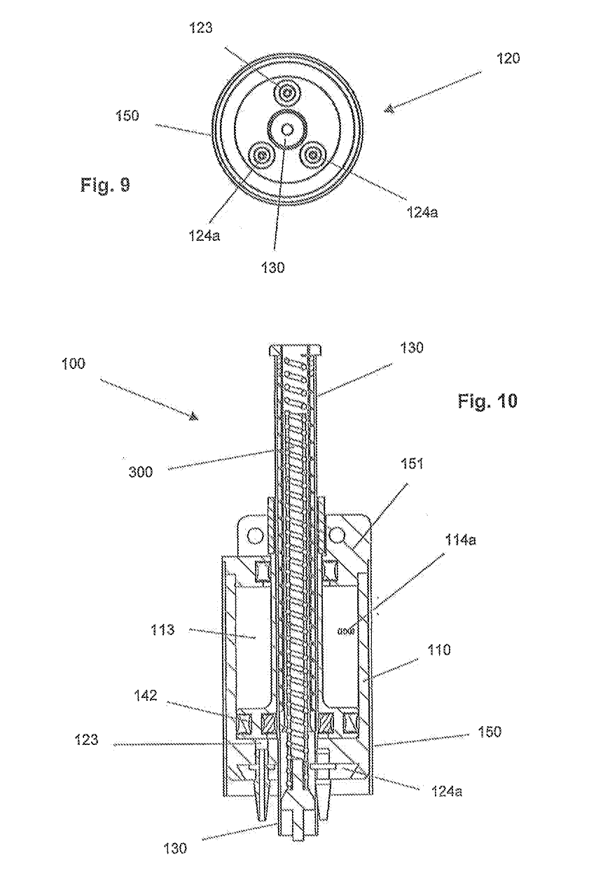

FIG. 9 shows a plan view of the cylinder base from FIG. 6;

FIG. 10 and FIG. 11 show two sectional views of the hydraulic actuating mechanism in the assembled state;

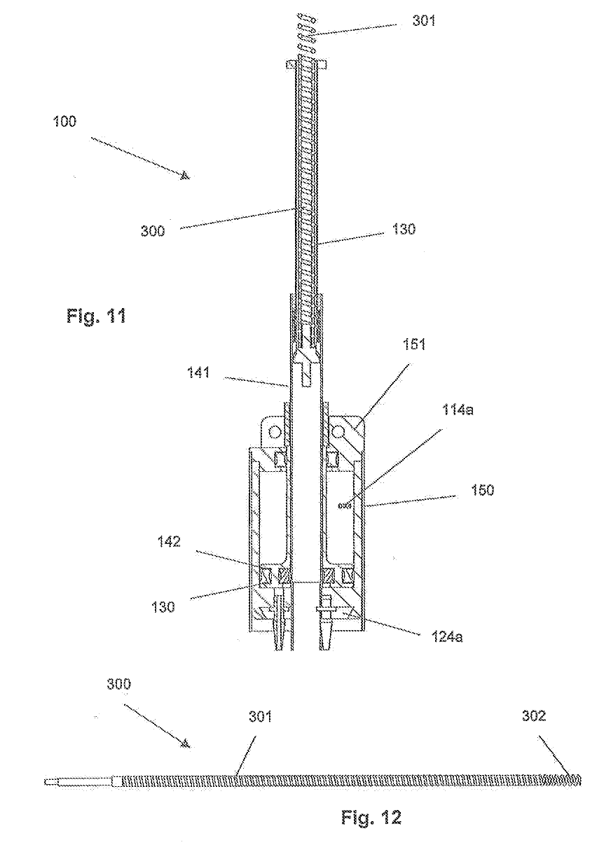

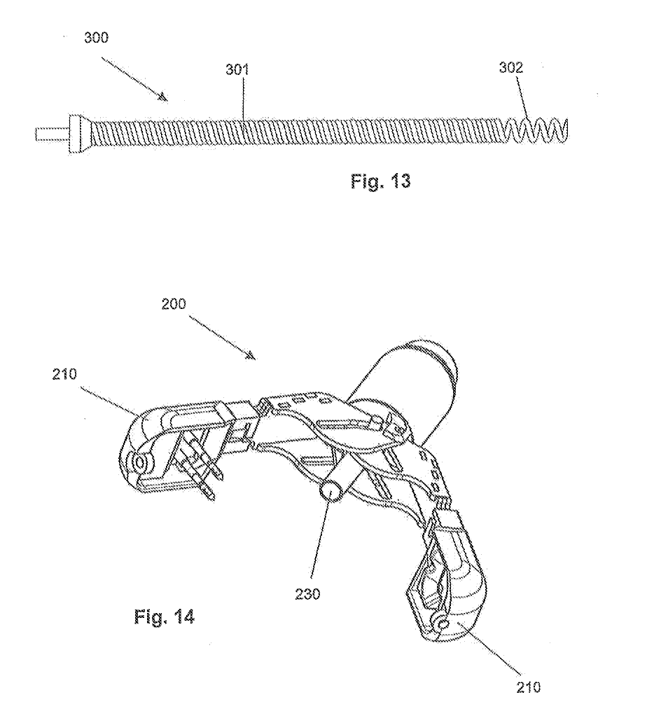

FIG. 12 and FIG. 13 show two embodiments of a tissue intervention element and

FIG. 14 shows an end effector for use in conjunction with the hydraulic actuating mechanism,

FIGS. 1 and 2 each show an exploded view of the hydraulic actuating mechanism 100 for an endosurgical and/or endoscopic instrument according to the invention. Provided here is a cylinder body 110 which is designed as a hollow cylinder, where overflow channels 112a, 112b, 112c are arranged on the outer wall 111 thereof. The cylinder base 120 is formed by a sealing disk which is arranged sealingly on the cylinder body 110.

The sealing disk forming the cylinder base 120 furthermore has an annular stepped edge 121 on the flank of which passage openings 122a, 122b, 122c for passage of the hydraulic fluid are arranged. These passage openings 122a, 122b, 122c are connected to the overflow channels 112a, 112b, 112c when the hydraulic actuating mechanism 100 according to the invention is assembled according to FIG. 4.

Furthermore the hydraulic actuating mechanism 100 has a piston 140 in the piston rod 141 whereof a passage 130 can be arranged. In the assembled state the piston skirt 142 is arranged sealingly in the interior 113 of the cylinder body 110 and can be moved inside the cylinder body 110 along its longitudinal axis A by introducing hydraulic fluid into the cylinder interior 113. In the assembled state the passage 130 passes through the cylinder base 120 and the cylinder interior 113 along the longitudinal axis A of the cylinder body 110 (FIG. 4).

According to FIG. 3 which shows the surface of the cylinder base 120 facing away from the cylinder interior 113, an inlet opening 123 for introducing hydraulic fluid into the cylinder interior 113 and outlet openings 124a, 124b, 124c, 124d, 124e which are connected to the overflow channels 112a, 112b, 112c are arranged on the cylinder base 120.

Finally the hydraulic actuating mechanism 100 has a sleeve 150 which in the assembled state is inverted over the cylinder body 110 and is arranged sealingly on the cylinder base 120 so that the overflow channels 112a, 112b, 112c are sealed against the environment. Provided on this sleeve 150 are connecting elements 151 on which an end effector 200—as shown for example in FIG. 14—can be arranged.

FIG. 4 shows the hydraulic actuating mechanism 100 according to the invention in the assembled state in a sectional view whilst FIG. 5 shows the cylinder body 110 in an oblique view. For actuating the hydraulic actuating mechanism 100 hydraulic fluid is introduced into the cylinder body 110 via the inlet opening 123 so that the piston 140 is raised. In order to bring the piston 140 into a predetermined defined position, one of the outlet openings 124a, 124b, 124c, 124d, 124e is released by opening a hydraulic valve so that the designed piston height which corresponds to a defined operating state of the end effector 200 is achieved due to the outflow of hydraulic fluid from the cylinder interior 113 through the outlet opening 114c where the hydraulic fluid then flows through the overflow channel 112c to the passage opening 122c and finally through the outlet opening 124c.

FIGS. 6 to 11 show another embodiment of the hydraulic actuating mechanism 100 according to the invention.

The hydraulic actuating mechanism 100 again has a cylinder body 110 which in this embodiment of the invention has two overflow channels 112a, 112b each having four outlet openings 114a, 114b for the hydraulic fluid, The cylinder base 120 is designed in one piece with the cylinder body 110 so that no additional sealing disk is required as in the previously described variant. The inlet opening 123 and the outlet openings 124a, 124b are arranged in the cylinder base 120 and are in direct fluid communication with the cylinder interior 113 or the overflow channels 112a, 112b (FIG. 8 and FIG. 9).

The piston rod 141 is again designed to be hollow where again a passage 130 is movable in the interior thereof along the longitudinal axis A of the cylinder body 110 with the aid of a guide element, for example, a spring wire (not shown). In the embodiment of the invention shown a tissue intervention element 300 with a corkscrew-like tip 301 is arranged in this passage 130 as shown in FIG. 12 and FIG. 13 for intervention in tissue.

In this embodiment of the invention the connecting element 151 for an end effector 200 is configured as a sealing disk through which the passage 130 is guided and which, in the assembled state of the hydraulic actuating mechanism 100 with the sleeve 150 sealingly terminates the cylinder body.

FIGS. 10 and 11 show the hydraulic actuating mechanism 100 in the assembled state in each case in a sectional view, where this is located completely inside the passage 130 in FIG. 10 whereas in FIG. 11 it is located in the extended position. The movement of the tissue intervention element 300 is accomplished independently of the actuation of the hydraulic system and is accomplished in a manner known per se by mechanical means.

In a first embodiment (FIG. 12) the tissue intervention element 300 comprises a flexible shaft 301 on which the corkscrew-like tip 302 is arranged in a free-standing manner.

In use in an endosurgical instrument this tissue intervention element 300 is guided through a passage 230 in an end effector 200 (FIG. 11) in a manner known per se when the end effector 200 is arranged on the connecting element 151 of the hydraulic actuating mechanism 100 according to the invention. The tissue can thus be held in position with the aid of the corkscrew-like tip 302 of the tissue intervention element 300 before the gripping elements 210 of the end effector 200 are closed around the tissue thus fixed in order, for example, to apply a seam to the tissue.

It is understood that the invention is not restricted to the exemplary embodiments shown. In particular the number and position of the overflow channels can be different, likewise different instruments can be arranged in the passage of the hydraulic actuating mechanism and most diverse end effectors can be connected to the connecting elements.

1. A medical instrument with an elongated element and an end effector arrangeable at one end of the elongated element, wherein the end effector is operable by means of a hydraulic actuating mechanism, the hydraulic actuating mechanism has a cylinder body with at least one, preferably two, three, four or five overflow channels for a hydraulic fluid, which are connected via at least one bore to the interior of the cylinder body, a piston arranged in the cylinder body and movable along the longitudinal axis of the cylinder body is provided, wherein the piston subdivides the interior of the cylinder body into a first cylinder chamber and a second cylinder chamber and is connected via a piston rod to the end effector, and at least one connection for introducing hydraulic fluid into the first cylinder chamber and at least one further connection which is connected to at least one overflow channel of the cylinder body are provided, wherein at least one passage for a surgical and/or optical and/or therapeutic instrument is arranged in the interior of the cylinder body.

2. The medical instrument according to claim 1, wherein the passage can be arranged in the piston rod.

3. The medical instrument according to claim 1, wherein the surgical instrument is a tissue intervention element, preferably having a corkscrew-like tip.

4. The medical instrument according to claim 1, wherein the at least one overflow channel is configured as a recess on the outer wall of the cylinder body and is connected via at least one bore to the interior of the cylinder body and the at least one overflow channel is connected to at least one connection for the hydraulic fluid.

5. The medical instrument according to claim 1, wherein the at least one overflow channel has a first section which runs as a recess on the outer wall of the cylinder body substantially normally to the longitudinal axis of the cylinder body and is connected via at least one bore to the interior of the cylinder body, the at least one overflow Channel has a second section which runs as a recess on the outer surface of the cylinder body substantially parallel to the longitudinal axis of the cylinder body and the at least one overflow channel has a third section in the base of the cylinder body which is connected to a connection for the hydraulic fluid.

6. The medical instrument according to claim 5, wherein the at least one bore of the first section of the at least one overflow channel is arranged between an upper and a lower piston stop so that the piston inside the cylinder body can be brought into at least one defined middle position which is arranged between the upper and the lower piston stop.

7. The medical instrument according to claim 1, wherein the end effector has two preferably claw-like gripping elements which can be moved towards one another from an open position into at least one closed position, preferably into two closed positions.
